(19) United States
(12) Patent Application Publication
(10) Pub. No.: US 2004/0209247 A1
(43) Pub. Date: Oct. 21, 2004

(54) E1-revertant-free adenoviral composition

(75) Inventors: Douglas E. Brough, Gaithersburg, MD (US); Imre Kovesdi, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 10/844,133

(22) Filed: May 12, 2004

Related U.S. Application Data
(63) Continuation of application No. 10/001,097, filed on Nov. 2, 2001.

(51) Int. Cl. C12Q 1/70

(52) U.S. Cl. 435/5

(57) ABSTRACT

The invention provides a composition comprising particles of an adenoviral vector comprising deficiencies in two or more gene functions required for viral replication, wherein at least one of the deficiencies is of a gene function of the E1 region of the adenoviral genome and (b) a carrier therefor, with relatively high ratios of (i) the number of particles of the adenoviral vectors to the number of particles of E1-revertant replication-deficient adenoviral vectors not comprising one or more of the deficiencies in gene functions of the E1 region of the adenoviral and (ii) the number of particles of the adenoviral vectors to the number of particles of replication-competent adenoviral vectors, as well as a method of preparing such a composition.

### CROSS-REFERENCE TO RELATED APPLICATIONS

This patent application is a continuation of copending U.S. patent application Ser. No. 10/001,097, filed Nov. 2, 2001.

### FIELD OF THE INVENTION

The present invention relates to a composition of adenoviral vectors that is substantially free of E1-revertant adenoviral vectors and a method of producing same.

### BACKGROUND OF THE INVENTION

One of the most efficient means of delivering a nucleic acid sequence to a cell is by placing the nucleic acid sequence into a viral vector and transfecting the cell with the viral vector. Suitable viral vectors for such use continue to be developed. Active viruses must be modified to reduce or eliminate viral-associated toxicity before use in vivo as viral vectors. Use of an active virus in a patient can result in uncontrolled secondary infection and/or inflammation, as well as undesired propagation of the virus. Thus, most viral vectors utilized for gene transfer have been rendered replication-deficient. However, the reversion of a replication-deficient viral vector to an infectious viral particle, in whole or in part, during propagation of the viral vector during manufacturing or during use in vivo remains an obstacle to the widespread acceptance of viral vectors as gene transfer vehicles.

Adenoviral vectors, for example, are made replication-deficient by removing or mutating one or more of the adenoviral genes necessary for replication. Most adenoviral vectors used for in vivo gene transfer are deleted in one or more of the coding sequences of the “early” region of the adenoviral genome, e.g., the E1, E2, and/or E4 regions. Since adenoviral vectors deficient in one or more gene functions essential for replication are unable to replicate autonomously, propagation of these adenoviral vectors for manufacturing (i.e. production) purposes must be performed in special cell lines developed to complement in trans the missing gene function(s). Alternatively, the replication-deficient viral vector is propagated in the presence of a helper virus, which provides in trans the missing function(s) required for viral replication (Berkner et al., J. Virol., 61, 1213-1220 (1987); Davidson et al., J. Virol., 61, 1226-1239 (1987); Mansour et al., Mol. Cell Biol., 6, 2684-2694 (1986)).

In some instances, propagation of replication-deficient vectors, e.g., E1/E3-deficient adenoviral vectors, in complementing cells or with helper virus can result in the formation of replication-competent viral vectors via homologous recombination between overlapping sequences in the cellular/helper virus genome and the genome of the replication-deficient viral vector (Lochmuller et al, Hum. Gene Ther., 5, 1485-1491 (1994); Hehir et al., J. Virol., 70, 8459-8467 (1997)). If, for example, the E1 region is reintroduced into an E1/E3 -deficient adenoviral viral vector through homologous recombination, the adenoviral vector becomes replication competent and is essentially an infectious molecule that can propagate freely, which is undesirable and can be detrimental to a patient.

In order to reduce the occurrence of replication-competent adenovirus (RCA), multiply deficient viral vectors (i.e., adenoviral vectors deficient in replication-essential gene functions in two or more regions of the adenoviral genome (e.g., the E1 and E4 regions)) were developed (see, for example, U.S. Pat. No. 5,994,106 and International Patent Application WO 95/34671). Multiply deficient adenoviral vectors can further reduce the possibility of generating RCA by requiring that two or more separate homologous recombination events occur to restore replication competence. The statistical chances of two recombination events are less than about 1 in 1×10¹⁰ (Zhu et al., Hum. Gene Ther., 10, 113-121 (1999)). However, the probability of homologous recombination still exists, although it is likely the event will not result in RCA. The re-introduction of viral sequences into the multiply deficient viral vector is still problematic with respect to purity of the viral vector composition from potentially harmful coding sequences. Thus, although multiply deficient adenoviral vectors can reduce the occurrence of RCA in an adenoviral vector population, undesirable recombination events which re-introduce viral sequences back into the viral genome but do not result in RCA nevertheless can occur.

Accordingly, there remains a need in the art for an adenoviral vector composition of increased purity with a reduced number of partially revertant replication-deficient adenoviral vectors relative to the replication-deficient adenoviral vectors of interest. The invention provides such a composition and a method of producing the composition. These and other advantages of the invention, as well as additional inventive features, will be apparent from the description of the invention provided herein.

### BRIEF SUMMARY OF THE INVENTION

The invention provides a composition comprising (a) at least 1×10⁴ particles of an adenoviral vector comprising an adenoviral genome comprising deficiencies in two or more gene functions required for viral replication, wherein at least one of the deficiencies is of a gene function of the E1 region of the adenoviral genome and (b) a carrier therefor. The ratio of the number of particles of the adenoviral vector to the number of particles of E1-revertant replication-deficient adenoviral vectors not comprising one or more of the deficiencies in gene functions of the E1 region of the adenoviral genome is greater than 1×10⁶:1, and the ratio of the number of the particles of adenoviral vector to the number of particles of replication-competent adenoviral vectors is greater than 1×10⁷:1.

The invention also provides a method of propagating an adenoviral vector. The method comprises providing an adenoviral vector comprising an adenoviral genome comprising deficiencies in two or more gene functions required for viral replication, wherein at least one of the deficiencies is of a gene function of the E1 region of the adenoviral genome, providing one or more cells that complement in trans for the deficiencies in the gene functions of the adenoviral vector, infecting one or more of the cells with the adenoviral vector, and culturing the cells in a medium so as to propagate at least 1×10⁴ particles of the adenoviral vector in the medium such that the ratio of the number of particles of the adenoviral vector to the number of particles of E1-revertant replication-deficient adenoviral vectors not comprising one or more of the deficiencies in gene functions of the E1 region of the adenoviral genome is greater than about 1×10⁶:1, and the ratio of the number of particles of the adenoviral vector to the number of particles of replication-competent adenoviral vectors is greater than about 1×10⁷:1.

The invention also provides a method of producing a replication-deficient adenoviral vector, wherein the method comprises propagating an adenoviral vector comprising an adenoviral vector genome comprising deficiencies in one or more gene functions required for viral replication in a cell that complements in trans for the gene function deficiencies and comprises a nucleic acid sequence encoding a non-complementation factor that downregulates the activity of cellular factors associated with homologous recombination.

The invention further provides a method of detecting an E1-revertant adenoviral vector in a composition comprising providing an adenoviral vector composition comprising particles of an adenoviral vector comprising an adenoviral genome comprising deficiencies in two or more gene functions required for viral replication, wherein at least one of the deficiencies is of a gene function of the E1 region of the adenoviral genome, providing one or more cells that complement in trans for the deficiencies in the gene functions of the adenoviral vector but do not complement in trans for the deficiency of a gene function of the E1 region, contacting the cells with the adenoviral vector composition, culturing the cell(s) in a medium, and determining whether any adenoviral vector propagation has occurred, which would be indicative of the presence of an E1-revertant adenoviral vector in the adenoviral vector composition.

### DETAILED DESCRIPTION OF THE INVENTION

Multiply deficient adenoviral vectors are desirable for effecting gene transfer to cells in that the probability of generating RCA is low during the manufacturing process and during use of the adenoviral vectors. In that respect, it is desirable to obtain a composition comprising a multiply deficient adenoviral vector wherein a deleted portion of the viral genome has not been re-introduced back into the vector backbone. In the case of an E1-deleted multiply deficient vector, a single recombination event in the E1 region will not restore replication competence, but can restore production of the E1 protein, which is known to be detrimental to eukaryotic cells. Moreover, if a heterologous nucleic acid sequence is inserted in the E1 region, the heterologous nucleic acid sequence can be replaced upon the occurrence of such a homologous recombination event. Depending on the particular gene function deficiencies of a replication-deficient adenoviral vector, a particular recombination event regarding the E1 region may or may not result in the generation of RCA. Typical screening procedures for RCA will not detect the presence of the E1 region in the adenoviral vector in the absence of the adenoviral vector being a RCA. Furthermore, any E1-producing virus in an E1-deficient adenoviral vector composition can potentially act as a helper virus for the replication of other E1-deficient viruses in the solution. Finally, E1-revertants grow more readily than E1-deficient vectors, giving them a selective advantage during propagation. (Lochmuller et al., Hum. Gene Ther., 5, 1485-1491 (1994)). The invention provides a replication-deficient adenoviral vector composition with a reduced occurrence of E1-revertant adenoviral vectors.

In particular, the composition comprises at least about 1×10⁴ particles of an adenoviral vector (also referred to as the adenoviral vector of interest) in a carrier therefor. The adenoviral vector comprises an adenoviral genome comprising deficiencies in two or more gene functions required for viral replication, and at least one of the deficiencies is of a gene function of the E1 region. The ratio of the number of particles of the adenoviral vector to the number of particles of E1-revertant replication-deficient adenoviral vectors not comprising one or more of the deficiencies in gene functions of the E1 region of the adenoviral genome is greater than about 1×10⁶:1, and the ratio of the number of particles of the adenoviral vector to the number of particles of replication-competent adenoviral vectors is greater than about 1×10⁷:1.

The adenoviral vector comprises an adenoviral genome deficient in two or more gene functions required for viral replication, wherein at least one of the deficiencies is of a gene function of the E1 region. By “region” is meant a fragment of the adenoviral genome, such as an early region (e.g., E1, E2, E3, or E4) or a late region (L1, L2, L3, L4, or L5), which is commonly associated with one function of the adenovirus, e.g., attachment, penetration, uncoating, replication, or formation of a structural protein. A “gene function” is a biological activity coded for by one or more nucleic acid sequences of the adenoviral genome. A function can be encoded by one or more regions of the adenoviral genome and a region can encode one or more gene functions. Each region of the adenoviral genome can contain nucleic acid sequences coding for more than one peptide, and/or a region can comprise nucleic acid sequences that encode RNA that is spliced to produce multiple different peptides from a single coding sequence.

The adenoviral vector can be any suitable adenoviral vector. Adenovirus (Ad) is a 36 kb double-stranded DNA virus that efficiently transfers DNA in vivo to a variety of different target cell types. The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Given that the human adenovirus serotype 5 (Ad5) genome has been completely sequenced, the adenoviral vector is described herein with respect to the Ad5 serotype. The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. Preferably, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

Preferably, the adenoviral vector is replication-deficient. By “replication-deficient” is meant that the adenoviral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient adenoviral vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). Preferably, the replication-deficient adenoviral vector comprises an adenoviral genome deficient in two or more gene functions required for viral replication. The two or more regions of the adenoviral genome are preferably selected from the group consisting of the E1, E2, and E4 regions. More preferably, the replication-deficient adenoviral vector comprises a deficiency in at least one replication-essential gene function of the E1 region (denoted an E1-deficient adenoviral vector). The E1 region of the adenoviral genome comprises the E1A region and the E1B region. The E1A and E1B regions comprise nucleic acid sequences coding for multiple peptides by virtue of RNA splicing. A deficiency of a gene function encoded by either or both of the E1A and/or E1B regions of the adenoviral genome (e.g., a peptide that performs a function required for replication) is considered a deficiency of a gene function of the E1 region in the context of the invention. In addition to such a deficiency in the E1 region, the recombinant adenovirus also can have a mutation in the major late promoter (MLP), as discussed in International Patent Application WO 00/00628. More preferably, the vector is deficient in at least one replication-essential gene function of the E1 region and at least part of the nonessential E3 region (e.g., an Xba I deletion of the E3 region) (denoted an E1/E3-deficient adenoviral vector).

Preferably, the adenoviral vector is “multiply deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Alternatively, the adenoviral vector lacks replication-essential gene functions in all or part of the E1 region and all or part of the E2 region (denoted an E1/E2-deficient adenoviral vector). Adenoviral vectors lacking replication-essential gene functions in all or part of the E1 region, all or part of the E2 region, and all or part of the E3 region also are contemplated herein. If the adenoviral vector is deficient in a replication-essential gene function of the E2A region, the vector preferably does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that the multiply replication-deficient adenoviral vector contain this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient adenoviral vectors, including multiply deficient adenoviral vectors, are disclosed in U.S. Pat. Nos. 5,851,806 and 5,994,106 and International Patent Applications WO 95/34671 and WO 97/21826. An especially preferred adenoviral vector for use in the present inventive method is that described in International Patent Application PCT/US01/20536.

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509.

The adenoviral vector, when multiply replication-deficient, especially in replication-essential gene functions of the E1 and E4 regions, preferably includes a spacer element to provide viral growth in a complementing cell line similar to that achieved by singly replication deficient adenoviral vectors, particularly an adenoviral vector comprising a deficiency in the E1 region. A spacer sequence is defined in the invention as any sequence of sufficient length to restore the size of the adenoviral genome to approximately the size of a wild-type adenoviral genome, such that the adenoviral vector is efficiently packaged into viral particles. The spacer element can contain any sequence or sequences which are of the desired length. The spacer element sequence can be coding or non-coding and native or non-native with respect to the adenoviral genome, but does not restore the replication-essential function to the deficient region. The spacer can be of any suitable size, desirably at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs. The size of the spacer is limited only by the size of the insert that the adenoviral vector will accommodate (e.g., approximately 38 base pairs). In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. The use of a spacer in an adenoviral vector is described in U.S. Pat. No. 5,851,806.

An adenoviral vector can contain any possible combination of spacer sequences and/or desired heterologous nucleic acid sequences (i.e., heterologous nucleic acid sequences as described above, for example, heterologous nucleic acid sequence(s) that preferably encodes a biologic activity in a host cell and can encode a peptide) in any combination of regions. The spacer sequence(s) and heterologous nucleic acid sequence(s) can be in the different regions or can be in the same region. For example, the adenoviral vector can contain a heterologous nucleic acid sequence in the E1 region and a spacer sequence in the E4 region, or a heterologous nucleic acid sequence in the E1 region and a spacer sequence in the E3 and/or E4 region, or a heterologous nucleic acid sequence in the E4 region and a spacer sequence in the E1 region, or a heterologous nucleic acid sequence in the E3 and/or E4 region and a spacer sequence in the E1 region, or a heterologous nucleic acid sequence in the E1, E2, E3, and E4 regions, or a heterologous nucleic acid sequence in the E1, E2, E3, and E4 regions with a spacer or spacers upstream and/or downstream of the heterologous nucleic acid.

The adenoviral vector preferably contains a packaging domain. The packaging domain can be located at any position in the adenoviral genome, so long as the adenoviral genome is packaged into adenoviral particles. Preferably, the packaging domain is located downstream of the E1 region. More preferably, the packaging domain is located downstream of the E4 region. In a particularly preferred embodiment, the replication-deficient adenoviral vector lacks all or part of the E1 region and the E4 region. In this preferred embodiment, a spacer is inserted into the E1 region, a desired heterologous nucleic acid sequence (e.g., a nucleic acid sequence encoding TNF-α) is located in the E4 region, and the packaging domain is located downstream of the E4 region. Thus, by relocating the packaging domain, the amount of potential overlap between the adenoviral vector and the cellular/helper virus genome is reduced.

The coat proteins of the adenoviral vector can be manipulated to alter the binding specificity of the resulting adenoviral particle. Suitable modifications to the coat proteins include, but are not limited to, insertions, deletions, or replacements in the adenoviral fiber, penton, pIX, pIIIa, pVI, or hexon proteins, or any suitable combination thereof, including insertions of various native or non-native ligands into portions of such coat proteins. Examples of adenoviral vector particles with modified binding specificity are described in, e.g., U.S. Pat. Nos. 5,871,727, 5,885,808, and 5,922,315. Preferred modified adenoviral vector particles include those described in, for example, Wickham et al., J. Virol., 71(10), 7663-9 (1997), Cripe et al., Cancer Res., 61(7), 2953-60 (2001), van Deutekom et al., J. Gene Med., 1(6), 393-9 (1999), McDonald et al., J. Gene Med., 1(2), 103-10 (1999), Staba et al., Cancer Gene Ther., 7(1), 13-9 (2000), Wickham, Gene Ther., 7(2), 110-4 (2000), Kibbe et al., Arch. Surg., 135(2), 191-7 (2000), Harari et al., Gene Ther., 6(5), 801-7 (2000), Bouri et al., Hum Gene Ther., 10(10), 1633-40 (1999), Wickham et al., Nat. Biotechnol., 14(11), 1570-3 (1996), Wickham et al., Cancer Immunol. Immunother., 45(3-4), 149-51 (1997), and Wickham et al., Gene Ther., 2(10), 750-6 (1995), and U.S. Pat. Nos. 5,559,099; 5,712,136; 5,731,190; 5,770,442; 5,801,030; 5,846,782; 5,962,311; 5,965,541; 6,057,155; 6,127,525; and 6,153,435; and International Patent Applications WO 96/07734, WO 96/26281, WO 97/20051, WO 98/07865, WO 98/07877, WO 98/40509, WO 98/54346, WO 00/15823, and WO 01/58940.

Construction of adenoviral vectors is well understood in the art. Adenoviral vectors can be constructed and/or purified using the methods set forth, for example, in U.S. Pat. No. 5,965,358 and International Patent Applications WO 98/56937, WO 99/15686, and WO 99/54441. The production of adenoviral vectors is well known in the art, and involves using standard molecular biological techniques such as those described in, for example, Sambrook et al., supra, Watson et al., supra, Ausubel et al., supra, and in several of the other references mentioned herein.

Replication-deficient adenoviral vectors are typically produced in complementing cell lines that provide gene functions not present in the replication-deficient adenoviral vectors, but required for viral propagation, at appropriate levels in order to generate high titers of viral vector stock. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only inverted terminal repeats (ITRs) and the packaging signal or only ITRs and an adenoviral promoter). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. The construction of complementing cell lines involves standard molecular biology and cell culture techniques, such as those described by Sambrook et al., supra, and Ausubel et al., supra. Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)).

The adenoviral vector can comprise a heterologous nucleic acid sequence. A “heterologous nucleic acid sequence” is a nucleic acid sequence that is not native to adenovirus. The adenoviral vector can comprise more than one heterologous nucleic acid sequence. The heterologous nucleic acid sequence can be an RNA, a peptide, or a polypeptide with a desired activity. Alternatively, the heterologous nucleic acid sequence can encode an antisense molecule or a ribozyme. The heterologous nucleic acid sequence preferably comprises a nucleic acid sequence encoding a protein (i.e., one or more nucleic acid sequences encoding one or more proteins). The nucleic acid sequence encoding the protein can be obtained from any source, e.g., isolated from nature, synthetically generated, isolated from a genetically engineered organism, and the like. An ordinarily skilled artisan will appreciate that any type of nucleic acid sequence (e.g., DNA, RNA, and cDNA) that can be inserted into a adenoviral vector can be used in connection with the invention. The heterologous nucleic acid sequence preferably encodes a biologic activity in a host cell and can encode a peptide such as a cancer therapeutic, an angiogenic factor, an anti-angiogenic factor, or a neurotrophic factor, or can comprise a nucleic acid sequence with activity in a cell (e.g., an RNA sequence, an antisense RNA sequence, and/or a ribozyme). The heterologous nucleic acid sequence can encode, for example, a member of the tumor necrosis factor super family of peptides (e.g., tumor necrosis factor-α (TNF-α), described in U.S. Pat. No. 4,879,226), a vascular endothelial growth factor (VEGF) (e.g., a non-heparin-binding VEGF, such as VEGF₁₂₁, VEGF₁₄₅, VEGF₁₆₅, VEGF₁₈₉, or VEGF₂₀₆, variously described in U.S. Pat. Nos. 5,332,671, 5,240,848, and 5,219,739), a pigment epithelium-derived factor (PEDF) or a derivative, (described in U.S. Pat. No. 5,840,686 and International Patent Applications 93/24529 and 99/04806), an atonal-associated factor (e.g., MATH-1 or HATH-1, described, e.g., in Birmingham et al., Science, 284, 1837-1841 (1999), and Zheng and Gao, Nature Neuroscience, 3(2), 580-586 (2000)), or an inducible nitric oxide synthase (iNOS) (described, e.g., in Yancopoulos et al., Cell, 93, 661-64 (1998) and references cited therein). The nucleic acid is preferably a secreted protein. By “secreted protein” is meant any peptide, polypeptide, or portion thereof, which is released by a cell into the extracellular environment. Additionally, the nucleic acid can encode a protein that affects splicing or 3′ processing (e.g., polyadenylation), or a protein that affects the level of expression of another gene within the cell (i.e., where gene expression is broadly considered to include all steps from initiation of transcription through production of a process protein), such as by mediating an altered rate of mRNA accumulation or transport or an alteration in post-transcriptional regulation.

The expression of the nucleic acid sequence encoding the protein is controlled by a suitable expression control sequence operably linked to the nucleic acid sequence. An “expression control sequence” is any nucleic acid sequence that promotes, enhances, or controls expression (typically and preferably transcription) of another nucleic acid sequence. Suitable expression control sequences include constitutive promoters, inducible promoters, repressible promoters, and enhancers. The nucleic acid sequence encoding the protein can be regulated by its endogenous promoter or, preferably, by a non-native promoter sequence. Examples of suitable non-native promoters include the cytomegalovirus (CMV) promoters, such as the CMV immediate-early promoter (described in, for example, U.S. Pat. No. 5,168,062), promoters derived from human immunodeficiency virus (HIV), such as the HIV long terminal repeat promoter, the phosphoglycerate kinase (PGK) promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, the sheep metallothionien promoter, the human ubiquitin C promoter, and the like. Alternatively, expression of the nucleic acid sequence encoding the protein can be controlled by a chimeric promoter sequence. The promoter sequence is “chimeric” in that it comprises at least two nucleic acid sequence portions obtained from, derived from, or based upon at least two different sources (e.g., two different regions of an organism's genome, two different organisms, or an organism combined with a synthetic sequence). Techniques for operably linking sequences together are well known in the art.

The promoter can be an inducible promoter, i.e., a promoter that is up- and/or down-regulated in response to an appropriate signal. For example, an expression control sequence up-regulated by a chemotherapeutic agent is particularly useful in cancer applications. The nucleic acid sequence preferably is operably linked to a radiation-inducible promoter, especially when the nucleic acid sequence encodes a TNF. The use of a radiation-inducible promoter provides control over transcription of the nucleic acid sequence, for example, by the administration of radiation to a cell or host comprising the adenoviral vector. Any suitable radiation-inducible promoter can be used in conjunction with the invention. The radiation-inducible promoter preferably is the early growth region-1 (Egr-1) promoter, specifically the CArG domain of the Egr-1 promoter. The Egr-1promoter is described in detail in U.S. Pat. No. 5,206,152 and International Patent Application WO 94/06916. The promoter can be introduced into the genome of the adenoviral vector by methods known in the art, for example, by the introduction of a unique restriction site at a given region of the genome. Alternatively, the promoter can be inserted as part of the expression cassette comprising the nucleic acid sequence coding for the protein, such as a TNF.

Preferably, the nucleic acid sequence encoding the protein further comprises a transcription-terminating region such as a polyadenylation sequence located 3′ of the region encoding the protein. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (human Sarcoma Virus-40) polyadenylation sequence.

The adenoviral vector can comprise a heterologous nucleic acid sequence in any suitable region of the adenoviral genome. The adenoviral vector can contain more than one heterologous nucleic acid sequence. In one embodiment, the heterologous nucleic acid sequences are located in separate regions of the adenoviral genome; however, the heterologous nucleic acid sequences can be placed next to each other, either upstream or downstream from one another, in the same region of the adenoviral genome. The heterologous nucleic acid sequence or sequences are preferably in a region of the adenoviral genome corresponding to a region wherein the adenoviral genome is deficient for a gene function required for viral propagation. For example, when the adenoviral vector is an E1-deficient adenovirus, the nucleic acid sequence encoding the protein is preferably located in the E1 region of the adenoviral genome. The insertion of a nucleic acid sequence into the adenoviral genome (e.g., the E1 region of the adenoviral genome) can be facilitated by known methods, for example, by the introduction of a unique restriction site at a given position of the adenoviral genome. The heterologous nucleic acid sequence can be inserted into, e.g., the E1 region, the E2 region, the E3 region, the E4 region, or any combination thereof.

An “E1-revertant” adenoviral vector in the context of the invention is an adenoviral vector that is not deficient in one or more of the gene functions of the E1 region that represent deficiencies in the abovementioned adenoviral vector (i.e., the adenoviral vector of interest in the composition). As discussed above, the adenoviral vector of the interest in the composition is deficient in two or more gene functions required for replication, at least one of which is coded by the E1 region. In an E1-revertant adenoviral vector, a deficient gene function of the E1 region has been restored by, for example, re-introduction of all or part of the E1 region previously removed or mutated to assist in rendering the adenoviral vector replication deficient. An E1-revertant adenoviral vector can be deficient in a gene function of one E1 region (e.g., the E1A region) but not deficient in a gene function of another E1 region (e.g., the E1B region). The E1-revertant adenoviral vector typically arises through a homologous recombination event in the E1 region between the adenoviral vector and nucleic acid sequences of a cellular/helper virus genome. An “E1-revertant replication-deficient” adenoviral vector is an E1-revertant adenoviral vector which does not have ability to replicate, i.e. is not an RCA, although the adenoviral vector is not deficient in at least one gene function, and perhaps all gene functions, of the E1 region.

A homologous recombination event in the E1 region resulting in an E1-revertant adenoviral vector could not only result in the loss of the heterologous nucleic acid sequence in the vector, but likely would have a detrimental effect on a host cell infected with the adenoviral vector. The E1 proteins are powerful transcriptional activators that induce viral replication by activating the cell replication cycle in host cells. In doing so, the E1 proteins allow the adenovirus to take advantage of the host cell machinery to replicate the viral genome (Shenk et al., Adv. Cancer Res., 57, 7-85 (1991); Nevins, Science, 258, 424-429 (1992)). Therefore, E1 proteins are oncogenes that transform normal cells into neoplastic cells. The E1 A protein has been linked to cellular transformation in vitro in cell cultures and in vivo in rodents (reviewed in Bayley et al., Int. J. Oncol., 5, 425-444 (1994)). Furthermore, E1A proteins are highly toxic to cells and, in some instances, instigate cell death through apoptosis, as well as enhancing cell killing by other agents, e.g., natural killer cells, macrophages, and cytokines such as tumor necrosis factor (Querido et al. J. Virol., 71, 3526-3533 (1997); Routes et al., Virology, 277, 48-57 (2000); Routes et al., J. Immunol., 165, 4522-4527 (2000)).

The adenoviral vector composition can be characterized by two ratios. The ratio of (a) the number of particles of the adenoviral vector comprising an adenoviral genome comprising deficiencies in two or more gene functions required for viral replication, wherein at least one of the deficiencies is of a gene function of the E1 region (i.e., the number of particles of the adenoviral vector of interest), to (b) the number of particles of E1-revertant replication-deficient adenoviral vectors not comprising (i.e., lacking) one or more of the deficiencies in gene functions of the E1 region of the adenoviral genome is greater than 1×10⁶:1, preferably greater than 1×10⁷:1, more preferably greater than 1×10⁸:1, and most preferably greater than 1×10⁹:1 or even greater than1×10¹⁰:1. The ratio of (a) the number of particles of the adenoviral vector comprising an adenoviral genome comprising deficiencies in two or more gene functions required for viral replication, wherein at least one of the deficiencies is of a gene function of the E1 region (i.e., the number of particles of the adenoviral vector of interest), to the number of particles of replication-competent adenoviral vectors in the composition is greater than 1×10⁷:1, preferably greater than 1×10⁸:1, preferably greater than 1×10⁹:1, and most preferably greater than 1×10¹⁰:1.

The presence of E1-revertant adenoviral vectors in a composition can be detected by any suitable technique known in the art for determining the presence of the nucleotide sequence(s) corresponding to the gene functions of interest in the adenoviral vectors of the composition. Suitable techniques include, for example, polymerase chain reaction (PCR), southern blotting, or a biological function assay. Preferably, the presence of E1-revertant adenoviral vectors in a composition is detected by a biological function assay, such as the method of detecting an E1-revertant adenoviral vector provided by the invention. A biological function assay is a method of detecting an E1-revertant adenoviral vector by inoculating a cell line that complements for every deficient gene function in the adenoviral vector except for the gene function(s) of the E1 region of interest. Thus, only adenoviral vectors that comprise the E1 region of interest (E1-revertants) will propagate in the cell line since the cell line does not complement for the E1 region of interest and any adenoviral vectors that are deficient in a gene function of the E1 region will not propagate. Cell culture and inoculation can be done using standard molecular biology techniques known in the art. (See, e.g., Maniatis et al., Molecular Cloning: A Laboratory Manual, (2d ed.), Cold Spring Harbor Press (1992), Watson et al., Recombinant DNA, (2d ed.), Scientific American Books (1992), Ausubel et al., Current Protocols in Molecular Biology (1987)).

The presence of E1-revertant adenoviral vectors in a composition also can be detected using PCR. Primers can easily be developed specific for the E1 gene functions deficient in the adenoviral vector of the composition. The composition can be purified, or the composition can be treated with a protease (e.g., Proteinase K) and heat denatured. PCR techniques known in the art can be utilized to determine if the E1 sequence of interest is present in the composition. As a control, PCR can be performed on the sample using primers specific for a cellular gene, such as 18s ribosomal RNA, to determine the presence of host cell DNA contamination in the samples.

The composition of the invention comprises 1×10⁴ or more adenoviral particles (also known as particle units (pu)) in a carrier therefor (e.g., 1×10⁵ or more particles, 1×10⁶ or more particles, 1×10⁷ or more particles, 1×10⁸ or more particles, or 1×10⁹ or more particles). The composition desirably comprises 1×10¹⁰ or more particles, more preferably 1×10¹¹ or more particles, even more preferably 1×10¹² or more particles, and most preferably 1×10¹³ or more particles (e.g., 1×10¹⁴ or more particles or 1×10¹⁵ or more particles). The number of adenoviral particle units can be determined by total viral titer techniques or other techniques suitable for determining the total number of viral vector particles. An E1-revertant-free composition preferably contains less than about 1 particle of E1-revertant adenoviral vector in approximately 10⁹, more preferably 10¹⁰, 10¹¹, 10¹², or 10¹³ particles of an adenoviral vector in a carrier therefor. An E1-revertant-free composition preferably comprises about 10 ng or less of E1 protein per milliliter, more preferably about 8 ng of E1 protein per milliliter, even more preferably about 5 ng of E1protein per milliliter, most preferably about 3 ng of E1 protein per milliliter.

The invention further provides a method of propagating an adenoviral vector by (a) providing an adenoviral vector comprising an adenoviral genome comprising deficiencies in two or more gene functions required for viral replication, where at least one of the deficiencies is of a gene function of the E1 region of the adenoviral genome, (b) providing one or more cells that complement in trans for the deficiencies in the gene functions of the adenoviral vector, (c) infecting one or more of the cells with the adenoviral vector, and (d) culturing the cells so as to propagate at least 1×10⁴ particles of the adenoviral vector in the medium. The method requires that (i) the ratio of the number of particles of adenoviral vectors to the number of particles of E1-revertant replication-deficient adenoviral vector not comprising one or more of the deficiencies in gene functions of the E1 region of the adenoviral genome is greater than 1×10^{6:1}, and (ii) the ratio of the number of particles of the adenoviral vector to the number of particles of replication-competent adenoviral vectors is greater than 1×10⁷:1.

The adenoviral vector can be any suitable adenoviral vector, for example, any suitable adenoviral vector described herein. Preferably, the adenoviral vector is a multiply deficient adenoviral vector, such as a multiply deficient adenoviral vector as described above. The adenoviral vector desirably contains a heterologous nucleic acid sequence. The ratios of adenoviral vector to E1-revertant adenoviral vector and to RCA are as described above in relation to the inventive composition. Furthermore, the method can be applied to any number of adenoviral vector particles as described herein (e.g., 1×10⁴ particles or more, 1×10⁵ particles or more, 1×10⁶ particles or more, 1×10⁷ particles or more, 1×10⁸ particles more more, 1×10⁹ particles or more, 1×10¹¹ particles or more, 1×10¹² particles or more, 1×10¹³ particles or more, 1×10¹⁴ particles or more, or 1×10¹⁵ particles or more).

The cell(s) can be any suitable cell(s). The cell(s) used to propagate the adenoviral vector are preferably cells that complement for the gene functions missing in the adenoviral vector. Preferred cells or cell lines complement in trans for at least one or more gene functions of the gene functions comprising the E1, E2, and E4 regions of the adenoviral genome. Other cells or cell lines include those that complement adenoviral vectors that are deficient in at least one gene function from the gene functions comprising the late regions, those that complement for a combination of early and late gene functions, and those that complement for all adenoviral functions. Desirably, the cell or cell line utilized specifically complements for those functions that are missing from the adenoviral vector of interest. Examples of such cell lines include HEK-293 (Graham et al., Cold Spring Harbor Svmp. Quant. Biol., 39, 637-650 (1975)), W162 (Weinberg et al., Proc. Nat. Acad Sci., 80, 5383-5386 (1983)), and gMDBP (Klessig et al., Mol. Cell BioL, 4, 1354-1362 (1984); Brough et al., Virology, 190, 624-634 (1992)), A549 cells (ATCC No. CCL-185), IMR90 fibroblast cells (ATCC No. CCL-186) (see, e.g., Hay et al., Human Gene Ther., 10, 579-590 (1999)), H460 cells (ATCC No. HTB-177) (see, e.g., Lee et al., Int. J. Cancer, 88, 454-463 (2000)), and HCT116 cells (ATCC No. HCL-247) (see, e.g., Ries et al., Nature Medicine, 6, 1128-1133 (2000)), an NCI-H1299 (ATCC CRL-5803) cell, a Calu-1 cell (ATCC HTB-54), and an NCI-H460 (ATCC HTB-177) cell. Alternatively, the cell is preferably a HeLa cell (ATCC CCL-2) or an ARPE-19/HPV-16 cell (ATCC CRL-2502). Suitable cells also include renal carcinoma cells, CHO cells, KB cells, SW-13 cells, MCF7 cells, and Vero cells. Other suitable cell lines include, for example, lung carcinoma cell lines such as NCI-H2126 (American Type Culture Collection (ATCC) No. CCL-256), NCI-H23 (ATCC No. CRL-5800), NCI-H322 (ATCC No. CRL-5806), NCI-H358 (ATCC No. CRL-5807), NCI-H810 (ATCC No. CRL-5816), NCI-H1155 (ATCC No. CRL-5818), NCI-H647 (ATCC No. CRL-5834), NCI-H650 (ATCC No. CRL-5835), NCI-H1385 (ATCC No. CRL-5867), NCI-H1770 (ATCC No. CRL-5893), NCI-H1915 (ATCC No. CRL-5904), NCI-H520 (HTB-182), and NCI-H596 (ATCC No. HTB-178). Also suitable are squamous/epidermoid carcinoma cell lines that include HLF-a (ATCC No. CCL-199), NCI-H292 (ATCC No. CRL-1848), NCI-H226 (ATCC No. CRL-5826), Hs 284.Pe (ATCC No. CRL-7228), SK-MES-1 (ATCC No. HTB-58), and SW-900 (ATCC No. HTB-59), large cell carcinoma lines (e.g., NCI-H661 (ATCC No. HTB-183)), and alveolar cell carcinoma lines (e.g., SW-1573 (ATCC No. CRL-2170)). Examples of suitable cells also include human embryonic kidney (HEK) cells, human retinal cells, human embryonic retinal (HER) cells, human embryonic lung (HEL) cells, and ARPE-19 cells. Suitable cell lines are described, for example, in U.S. Pat. No. 5,994,106 and International Patent Application WO 95/34671. Particularly preferred cell lines include, for example, cell lines designated as 293/E4, 293/ORF-6, and 293/E4/E2A, which are described in U.S. Pat. Nos. 5,851,806 and 5,994,106. Additional appropriate cell lines can be generated using standard molecular biology techniques, such as those set forth in, e.g., Maniatis et al., Molecular Cloning: A Laboratory Manual, (2d ed.), Cold Spring Harbor Press (1992), Watson et al., Recombinant DNA, (2d ed.), Scientific American Books (1992), and Ausubel et al., Current Protocols in Molecular Biology (1987). In some embodiments, it may be desirable for the cell to contain a helper virus which complements for adenoviral gene functions required for viral replication that are not provided by the cell.

The cells form a culture that is maintained in a suitable medium. The culture of cells can be any culture suitable for the propagation of an adenoviral vector. Examples of suitable cultures include perfusion cultures, substrate-supported cultures, microcarrier-supported cultures, fluid bed cultures, and suspension cultures. The medium can be any medium appropriate for maintaining the cells and propagating an adenoviral vector or vectors therein. Mediums suitable for use in the invention, along with techniques used to develop new or modified mediums suitable for use in the context of the invention, are known in the art. In general, the medium will contain a selection of secreted cellular proteins, diffusible nutrients, amino acids, organic and inorganic salts, vitamins, trace metals, sugars, and lipids. The medium can also contain additional compounds such as growth promoting substances (e.g., cytokines). A suitable medium preferably has the physiological characteristics and conditions (e.g., pH, salt content, vitamin and amino acid profiles) under which the cells naturally flourish. Numerous commercial cell and medium combinations are available, and one of ordinary skill will readily be able to determine the desired conditions for the culture.

The culture can be prepared in any suitable manner that promotes the growth and sustenance of the cells. Typically the medium is inoculated with a cell or cells. After inoculation with the cells, the culture is then “cultured” or cultivated under conditions to permit growth of the cells. Any suitable manner of culturing the culture that permits the growth of the cells is suitable in the context of the invention. The method of culturing such cells will depend upon the type of adenoviral vector cell selected. Suitable culturing methods are well known in the art and typically involve maintaining pH and temperature within ranges suitable for growth of the cells. Preferred temperatures for culturing are about 35-40° C., more preferably about 36-38° C., and optimally about 37° C. Preferably, the pH of the culture is maintained at about 6-8, more preferably at about 6.7-7.8, and optimally at about 6.9-7.5.

The carrier of the composition comprising the adenoviral vector can be any suitable carrier for the adenoviral vector. Suitable carriers for the adenoviral vector composition are described in U.S. Pat. No. 6,225,289. The carrier typically will be liquid, but also can be solid, or a combination of liquid and solid components. The carrier desirably is a pharmaceutically acceptable (e.g., a physiologically or pharmacologically acceptable) carrier (e.g., excipient or diluent). Pharmaceutically acceptable carriers are well known and are readily available. The choice of carrier will be determined, at least in part, by the particular adenoviral vector and the particular method used to administer the composition. The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

Formulations suitable for oral administration include (a) liquid solutions, such as an effective amount of the active ingredient dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base (such as gelatin and glycerin, or sucrose and acacia), and emulsions, gels, and the like containing, in addition to the active ingredient, such excipients as are known in the art.

Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the adenoviral vector and physiological distress. Immune system suppressors can be administered with the composition method to reduce any immune response to the adenoviral vector itself or associated with a disorder. Alternatively, immune enhancers can be included in the composition to upregulate the body's natural defenses against disease. Moreover, cytokines can be administered with the composition to attract immune effector cells to the tumor site.

Anti-angiogenic factors, such as soluble growth factor receptors, growth factor antagonists, i.e., angiotensin, and the like, also can be part of the composition. Similarly, vitamins and minerals, anti-oxidants, and micronutrients can be co-administered with the composition. Antibiotics, i.e., microbicides and fungicides, can be present to reduce the risk of infection associated with gene transfer procedures and other disorders.

The invention also provides a method of propagating an adenoviral vector by (a) providing an adenoviral vector comprising an adenoviral genome comprising deficiencies in two or more gene functions required for viral replication, where at least one of the deficiencies is of a gene function of the E1 region of the adenoviral genome, (b) providing one or more cells that complement in trans for the deficiencies in the gene functions of the adenoviral vector, (c) infecting one or more of the cells with the adenoviral vector, and (d) culturing the cells so as to propagate at least 1×10⁴ or more particles of the adenoviral vector in the medium. The method requires that, as to the resulting medium containing the propagated adenoviral vector, (i) the ratio of the number of particles of adenoviral vectors to the number of particles of E1-revertant replication-deficient adenoviral vector not comprising one or more of the deficiencies in gene functions of the E1 region of the adenoviral genome is greater than 1×10⁶:1, and (ii) the ratio of the number of particles of the adenoviral vector to the number of particles of replication-competent adenoviral vectors is greater than 1×10⁷:1. The discussion of the amount of adenoviral vector particles and the aforementioned ratios with respect to the inventive composition equally apply to the inventive method, particularly the resulting medium thereof.

The inventive method provides that the cells produce less than about one E1-revertant adenoviral vector for at least about 20 passages after infection, (e.g., at least about 30, 40, 100, or more passages), the cells produce less than about one E1-revertant adenoviral vector in a period of about 36 hours after infection with the adenoviral vector (e.g., 24, 30, 42, or 48 hours), the cells produce less than about one E1-revertant adenoviral vector per 1×10¹⁰ particles of the adenoviral vector produced by the cells (preferably per 1×10¹¹ particles, more preferably per 1×10¹² particles, and most preferably per 1×10¹³ particles), or any combination of the above. Optimally, the amount of overlap (i.e., “region of homology”) between the cellular genome and the adenoviral genome (i.e., the genome of the adenoviral vector being propagated in the cell) or, if appropriate, the adenoviral genome and helper virus genome, is insufficient to mediate a homologous recombination event that results in an E1-revertant adenoviral vector. The region of homology is preferably less than about 2000 base pairs, preferably less than about 1000 base pairs (e.g., less than about 1500 base pairs), more preferably less than about 700 base pairs, and most preferably less than about 300 base pairs.

The invention also provides a method of producing a replication-deficient adenoviral vector, where an adenoviral vector comprising an adenoviral genome comprising deficiencies in one or more gene functions required for viral replication, in a cell that complements in trans for the gene function deficiencies and comprises a nucleic acid sequence encoding a non-complementation factor that reduces the rate of homologous recombination between nucleic acids in the cell (e.g., by downregulating the activity of cellular factors associated with homologous recombination). By “non-complementation factor” is meant a factor that does not complement for an adenoviral gene function required for viral replication (i.e., a replication-essential gene function) that is lacking from the replication-deficient adenoviral vector to be propagated. Thus, the non-complementation factor used will, in some instances be dictated by the particular adenoviral vector to be propagated. For example, open reading frame-6 (ORF-6) of the adenoviral E4 region is appropriate for use in the inventive method when the E4 region of the adenoviral vector is intact.

The non-complementation factor can be, but is not limited to, a bacterial factor, an adenoviral factor, a yeast factor, a mammalian factor, or a chemical compound. The non-complementation factor preferably is a factor encoded by a subgroup C adenoviral vector (e.g., Ad2 or Ad5). However, the adenoviral factor can be derived from a non-group C adenovirus or a non-human adenovirus. No matter the origin of the non-complementation factor, the non-complementation factor displays at least about 40% homology to human serotype 5 adenoviral E4 34 kD protein at the amino acid level. Ideally, the non-complementation factor that reduces the rate of homologous recombination between nucleic acids in the cell is any peptide that is more than about 40% homologous (preferably more than about 50% homologous, more preferably more than about 70% homologous, and most preferably more than about 80% homologous) to the Ad5 E4 34 kD protein at the amino acid level. The degree of amino acid homology can be determined using any method known in the art, such as the BLAST sequence database. Furthermore, a homolog of the Ad5 E4 34 kD protein can be any peptide which is encoded by a nucleic acid sequence that hybridizes to the nucleic acid sequence encoding the Ad5 E4 34 kD protein under at least moderate, preferably high, stringency conditions. Exemplary moderate stringency conditions include overnight incubation at 37° C. in a solution comprising 20% formamide, 5× SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1× SSC at about 37-50° C., or substantially similar conditions, e.g., the moderately stringent conditions described in Sambrook et al., supra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5× SSC (0.75 M NaCi, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2× SSC, (ii) at 55° C. in 50% formamide and (iii) at 55° C. in 0.1×SSC (preferably in combination with EDTA). Additional details and an explanation of stringency of hybridization reactions are provided in, e.g., Ausubel et al., supra. For example, a suitable non-human adenoviral factor can be derived from a porcine, murine, canine, or bovine adenovirus E4 region, which does not complement for an E4-deficient vector derived from human adenovirus serotype 5 or is used in conjunction with a replication-deficient adenoviral vector comprising an intact E4 region.

The non-complementation factor preferably acts on factors involved in homologous recombination such as, for example, bacterial RecA, RecG, RuvAB, and RuvC; yeast Rad51, Rad52, Rad54, Rad55, Rad57, Mre11, Rad50, and Xrs2; mammalian Ras51, Dmc1, Xrcc2, Xrcc3, Rad51B, Rad51C, and Rad51D; human Ub11, RecQ, BRCA1, BRCA2, p53, and ATM (reviewed in, for instance, Vasquez et al., Proc. Nat. Acad Sci., 98, 8403-8410 (2001)). Preferably, the non-complementation factor acts to downregulate homologous recombination. Downregulation of homologous recombination can be partial downregulation, since total downregulation of homologous recombination is not required to achieve the desired affect in the context of the invention.

The invention further provides a method of detecting an E1-revertant adenoviral vector in a composition. The method comprises providing an adenoviral vector composition comprising particles of an adenoviral vector comprising an adenoviral genome comprising deficiencies in two or more gene functions required for viral replication, wherein at least one of the deficiencies is of a gene function of the E1 region of the adenoviral genome, providing one or more cells that complement in trans for the deficiencies in the gene functions of the adenoviral vector but do not complement in trans for the deficiency of a gene function of the E1 region, contacting the cells with the adenoviral vector composition, culturing the cell(s) in a medium, and determining whether any adenoviral vector propagation has occurred, which would be indicative of the presence of an E1-revertant adenoviral vector in the adenoviral vector composition. The cells do not complement in trans for the deficiencies of the gene function of the E1 region, thus, only adenoviral vectors which have regained the gene function of the E1 region will propagate in the cell(s) of the inventive method. Suitable adenoviral vectors, cells, and culture conditions for use in the inventive method are described above.

The following example further illustrates the invention but, of course, should not be construed as in any way limiting its scope.

### EXAMPLE

This example demonstrates a biological function assay for testing for E1-revertant adenoviral vectors.

An E1 (+)E4(−) cell line was constructed by the stable transfection of A549 cells with an inducible E4-ORF6 construct. The cell line complements for deficiencies in gene functions of the E4 region but not for deficiencies in the E1 region of the adenoviral genome. The cells are infected with a composition of E1(−)E4(−) adenoviral vectors grown in a cell line that complements for the E1 and E4 deficiencies of the adenoviral vectors. Specifically, the cells are infected with a composition of the Ad_{Gv}11 vector grown in 293 cells complementing for the E1 and E4 deficiencies as described, for example, in International Patent Application WO 95/34671. The cells are cultured using routine tissue culture techniques. Only adenoviral vectors which have regained the gene function(s) of the E1 region will propagate in the cell line, and those vectors which remain E1 (−)E4(−) will not do so. Accordingly, detection of adenoviral vector propagation evidences the presence of E1-revertant adenoviral vectors in the composition.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of evaluating an adenoviral vector composition, wherein the composition comprises (a) at least 1×10⁴ particles of an adenoviral vector comprising an adenoviral genome, wherein the adenoviral vector is an human subgroup C adenoviral vector deficient in at least the E1 region of the adenoviral genome and the E4 region of the adenoviral genome, and (b) a carrier therefor, wherein the method comprises (i) evaluating the ratio of the number of particles of the adenoviral vector to the number of particles of E1-revertant replication-deficient adenoviral vectors not deficient in the E1 region of the adenoviral genome, wherein the ratio of the number of particles of the adenoviral vector to the number of particles of E1-revertant replication-deficient adenoviral vectors not deficient in the E1 region of the adenoviral genome is greater than 1×10⁶:1, and (ii) evaluating the ratio of the number of particles of the adenoviral vector to the number of particles of replication-competent adenoviral vectors, wherein the ratio of the number of particles of the adenoviral vector to the number of particles of the replication-competent adenoviral vector is greater than 1×10⁷:1.

2. The method of claim 1, wherein the composition comprises at least 1×10¹⁰ particles of the adenoviral vector.

3. The method of claim 2, wherein the ratio of the number of particles of the adenoviral vector to the number of particles of E1-revertant replication-deficient adenoviral vectors not deficient in the E1 region of the adenoviral genome is greater than 1×10⁷:1.

4. The method of claim 3, wherein the ratio of the number of particles of the adenoviral vector to the number of particles of E1-revertant replication-deficient adenoviral vectors not deficient in the E1 region of the adenoviral genome is greater than 1×10⁸:1.

5. The method of claim 4, wherein the ratio of the number of particles of the adenoviral vector to the number of particles of E1-revertant replication-deficient adenoviral vectors not deficient in the E1 region of the adenoviral genome is greater than 1×10⁹:1.

6. The method of claim 5, wherein the ratio of the number of particles of the adenoviral vector to the number of particles of E1-revertant replication-deficient adenoviral vectors not deficient in the E1 region of the adenoviral genome is greater than 1×11¹⁰¹⁰:1.

7. The method of claim 1, wherein the adenoviral vector is deficient in the E2 region of the adenoviral genome.

8. The method of claim 1, wherein the adenoviral vector is deficient in a late region of the adenoviral genome.

9. The method of claim 1, wherein the adenoviral vector is deficient in all regions required for viral replication.

10. The method of claim 9, wherein the adenoviral vector comprises at least one adenoviral inverted terminal repeat and one or more adenoviral promoters.

11. The method of claim 9, wherein the adenoviral vector comprises at least one adenoviral inverted terminal repeat and a packaging signal.

12. The method of claim 1, wherein the composition comprises 1×10¹ to 1×10¹³ particles of the adenoviral vector.

13. The method of claim 1, wherein the composition comprises about 10 ng/ml or less of E1 protein.

14. The method of claim 1, wherein the adenoviral vector comprises a heterologous nucleic acid sequence.

15. The method of claim 14, wherein the heterologous nucleic acid sequence is located in the E1 region.

16. The method of claim 14, wherein the heterologous nucleic acid sequence encodes tumor necrosis factor-α, a vascular endothelial growth factor, a pigment-epithelial derived factor, or an atonal-associated factor.

17. The method of claim 16, wherein the heterologous nucleic acid sequence encodes tumor necrosis factor-α.

18. The method of claim 14, wherein the heterologous nucleic acid sequence is located in the E1 region and/or the E4 region.

19. The method of claim 18, wherein the heterologous nucleic acid sequence encodes tumor necrosis factor-α, a vascular endothelial growth factor, a pigment-epithelial derived factor, or an atonal-associated factor.

20. The method of claim 1, wherein the adenoviral vector comprises a spacer sequence in the E4 region.

21. The method of claim 1, wherein the adenoviral vector comprises a packaging domain located downstream of the E1 region.

22. The method of claim 21, wherein the packaging domain is located downstream of the E4 region.

23. The method of claim 1, wherein the adenoviral vector is serotype 2.

24. The method of claim 1, wherein the adenoviral vector is serotype 5.

25. The method of claim 1, wherein step (i) comprises:
(i-a) providing one or more cells that complement in trans for the deficiencies in the adenoviral genome of the adenoviral vector, except for the deficiencies of the E1 adenoviral region,
(i-b) contacting one or more of the cells with a sample of the composition,
(i-c) culturing the cells in a medium, and
(i-d) determining whether an E1-revertant adenoviral vector is present in the composition by analyzing the propagation of any adenoviral vector.

26. The method of claim 25, wherein the adenoviral vector is serotype 2.

27. The method of claim 25, wherein the adenoviral vector is serotype 5.
